(19) United States
(12) Patent Application Publication
(10) Pub. No.: US 2007/0142838 A1
(43) Pub. Date: Jun. 21, 2007

(54) Surgical suture staple and attachment device for securing a soft tissue to a bone

(76) Inventor: Christopher Jordan, Midwest City, OK (US)

Correspondence Address: Randal D. Homburg, P.O. Box 10470, Midwest City, OK, 73140-1470, US

(21) Appl. No.: 11/311,099

(22) Filed: Dec. 19, 2005

(51) Int. Cl. A61F 2/30

(52) U.S. Cl. 606/75

(57) ABSTRACT

A surgical suture staple and an application appliance for the secure and permanent attachment of a soft tissue to a bone provides a staple having a first and second metal or composite pin which will not adversely affect the bone within which it is attached, the first and second pin connected by a synthetic fiber suture. At least one suture is swedged into each first pin near an upper pin head and either swedged into or adjustably attached through the second pin also near an upper pin head, the first and second pins further comprising an extendable securing means which prevents the first and second pin from being removed from the bone within which it is implanted and a depression within the upper pin head receiving the driving end of an application appliance. The first and second pins are driven into a bone slightly below the bone surface on each side of a soft tissue being anchored to the bone, the suture securing the soft tissue with or without penetration.

### CROSS REFERENCE TO RELATED APPLICATIONS

None

### I. BACKGROUND OF THE INVENTION

1. Field of Invention

A surgical suture staple and an application appliance for the secure and permanent attachment of a soft tissue to a bone provides a staple having a first and second metal or composite pin which will not adversely affect the bone within which it is attached, the first and second pin connected by a synthetic fiber suture. At least one suture is swedged into each first pin near an upper pin head and either swedged into or adjustably attached through the second pin also near an upper pin head, the first and second pins further comprising an extendable securing means which prevents the first and second pin from being removed from the bone within which it is implanted and a depression within the upper pin head receiving the driving end of an application appliance. The first and second pins are driven into a bone slightly below the bone surface on each side of a soft tissue being anchored to the bone, the suture securing the soft tissue with or without penetration.

2. Description of Prior Art

The following United States patents were discovered and are disclosed within this application for utility patent. All relate to surgical staples and suturing devices.

A hard and soft tissue closure device and method is disclosed in U.S. Pat. No. 6,485,504 to Johnson, which discloses a sleeve which is driven through a bone within which is inserted a suture or a staple or a two piece pin component. The penetrating device and the connecting devices are shown in multiple embodiments. A piton-like device providing an anchor point to a bone for a suture to be secured is disclosed in U.S. Pat. No. 6,200,330 to Benderev, which includes a t least two prongs that flare out into a direction perpendicular to the direction of insertion. U.S. Pat. No. 6,149,658 to Gardiner provides a suture staple apparatus for constructing a graft to artery anastomosis as well as application to other soft tissue anastomoses, the suture staple comprising a needle, a pin, a base and a flange, with the needle penetrating the soft tissue and the pin, base and flange provided to seal the graft and artery together. A great deal of the disclosure is dedicated to the mechanical instrument to apply the suture staple in addition to the actual suture staple.

Most similar, although patently distinct from the present invention, is a device for applying a meniscal staple, disclosed in U.S. Pat. No. 6,190,401 to Green, the staple being associated relevant prior art. However, that device is disclosed as having two needles having a sharp tip at one end and a blunt end within connecting leg members connected together by a flexible member, such as a suture, the anchor members having a plurality of barb-like projections, each barb having a tapered projection to allow for the anchor member to be pushed into the tissue, but restricts withdrawal of the anchor member once inserted. This is suture staple is also generally described as a reabsorbable meniscal staple. The remainder of the patent is devoted to disclosure of the mechanical device used to apply the suture staple, being a springloaded triggered staple gun with a pair of driving rods which exert a driving force to the blunt end of the suture staple, with the driving rods applying a uniform and common driving force to both blunt ends with equal force. The stated purpose of the device is to attache a menicus in a joint to a bone. It appears that the pins are withdrawn once the anchor members are inserted into the target tissue.

In addition to the above noted patents, several surgical anchors and sutures are disclosed in published advertisements most commonly found in orthopedic journals and periodicals, including products advertised by CONMED™. Additionally, use in the field of arthroscopic surgery for surgical anchors and sutures is indicated in numerous articles, including Intraoperative Arthroscopic Suture Anchor Reloading; Arthroscopy: The Journal of Arthroscopic and Related Surgery, Vol. 21, No. 7 (July), 2005: P 898, et.seq.

### II. SUMMARY OF THE INVENTION

The field of orthopedic surgery has made numerous advances in surgical appliances and instruments, especially in dealing with means to reattach torn soft tissues to bone or other soft tissues. In many cases, this reattachment requires some device which is implanted into a bone that connects to a suture which is used to tie the soft tissue to the bone, whether permanently or temporarily. Staples or anchors are generally used as the implant device attached or installed into the bone which either include pre-threaded sutures or locations for sutures to be subsequently attached. Methods of application of the implant device to the bone either require a mechanical “gun” or stapler or in the type anchors having an outer thread, a screwdriver or ratchet-type device or employ the use of a surgical hammer. Sutures are either passed through the implant device prior to their insertion or installation, already attached using a technique known in the art as “swedging” or the sutures are passed through ports, eyelets or sockets in the implant device subsequent to the insertion or installation.

In most cases, the sutures are disclosed as being inserted through the soft tissue, actually sewing the soft tissue to the implant device. Soft tissue can be torn and weakened during this suturing process, and in many cases, multiple sutures are required to disperse the stress forces of a single suture through soft tissue by requiring multiple penetrations of the soft tissue while attaching the soft tissue to the implant device, indicated in the prior art by multiple colored sutures in a singular implant device.

It would be useful in the field of arthroscopic surgery to provide a suture staple device which allows for the primary or secondary attachment of a soft tissue to a bone with and without penetration of the soft tissue. The present device provides at least one pair of penetrating bone pins joined together either by a fixed length of suture material or an adjustable length of suture material, the penetrating pins, provided in several useful embodiments having differing suture connections applied to the bone on either side of or sometimes through the soft tissue to be attached to the bone, with the suture material securing the soft tissue to the bone without or without necessary penetration of the soft tissue by the suture, to be used alone or in combination with other arthroscopic securing devices.

Several embodiments of the penetrating bone pins include a different means of suture attachment, some having one or more pre-attached permanently connected sutures, or having orifices which allow for simply passing a suture through the pin or having an orifice that locks the suture into a fixed position by tension after adjustments to the length of the suture connected to another pin has been made.

### III. DESCRIPTION OF THE DRAWINGS

The following drawings are submitted with this utility patent application.

FIG. 1 is a side view of the suture staple device with two single swedged pins and a fixed length suture.

FIG. 2 is a side view of the suture staple device with a single swedged pin and a passage suture pin with an adjustable length suture.

FIG. 3A is a side view of a double swedged pin with two sutures.

FIG. 3B is a side view of an adjustable locking passage suture pin.

FIG. 4 is a perspective view of the suture staple device and the application appliance for insertion of the suture staple device, with phantom lines indicating the outer insertion tube sleeve with projecting anchoring barbs.

FIG. 5 is a top view of the suture staple device within the outer insertion tube sleeve.

FIG. 6 is a cross-sectional view of the suture staple device and the end of a driving rod demonstrating the relationship between the securing aperture within the upper pin head of the pin and the securing projection on the driving end of the driving rod.

FIG. 7 is a sectional view of the suture staple device within a bone securing a segment of soft tissue without penetration.

FIG. 8A is a demonstration of an anatomical application of the suture staple device within a knee joint securing a segment of repaired soft tissue to a bone without penetration.

FIG. 8B is a demonstration of an anatomical application of the suture staple devices within a knee joint securing a segment of repaired soft tissue to a bone utilizing a combination of pins and at least one suture.

### IV. Description of the Preferred Embodiment

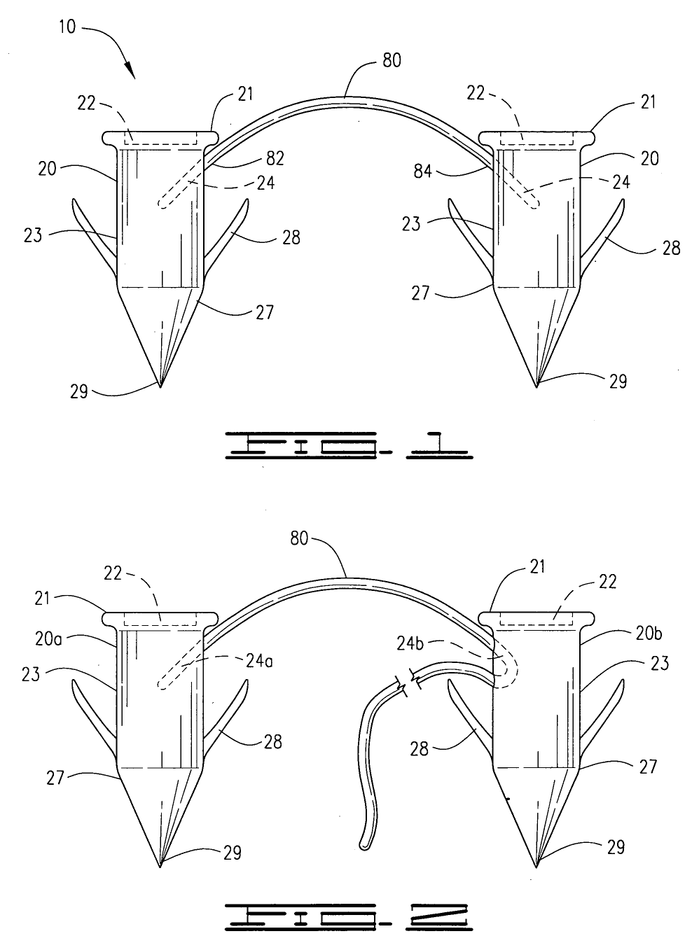

A suture staple device 10 for the primary or secondary attachment of a soft tissue 100 to a bone 200 with or without requiring penetration of the soft tissue, shown in FIGS. 1-8B of the drawings, comprises at least two biocompatible 20 pins and at least one biocompatible suture 80 attached between the at least two pins 20. Each pin 20 further comprises an expanded upper pin head 21 defining a securing aperture 22, a shaft 23 having at least one suture attachment orifice 24, a lower end 27 having two lateral projecting bone anchor fins 28, the lower end 27 defining a tapered point 29. The device 10 further includes an application appliance 40, FIGS. 4-6, comprising an outer insertion tube 42 having two parallel cylindrical insertion barrels 43 connected by a linear suture passage 44, the outer insertion tube 42 having an open upper end 45 and an open lower end 46, the lower end 46 having at least two projecting anchor barbs 48, the application appliance 40 further comprising two driving rods 50, each driving rod 50 having an upper end 52 defining an end cap 54 with an upper striking surface 55 and a lower end 56 terminating into a securing projection 58 adapted to securely fit within the securing aperture 22 of an upper pin head 21.

The pin 20 is provided in at least four embodiments, including a first embodiment referenced as a single swedged pin 20a, a second embodiment referenced as a passage suture pin 20b, a third embodiment referenced as a double swedged swedged pin 20c, and a fourth embodiment referenced as an adjustable locking passage suture pin 20d. Any combination of the four embodiments of the pins 20a-20d may be utilized, depending on the application need of the surgical repair to be conducted to attach soft tissue 100 to the bone 200.

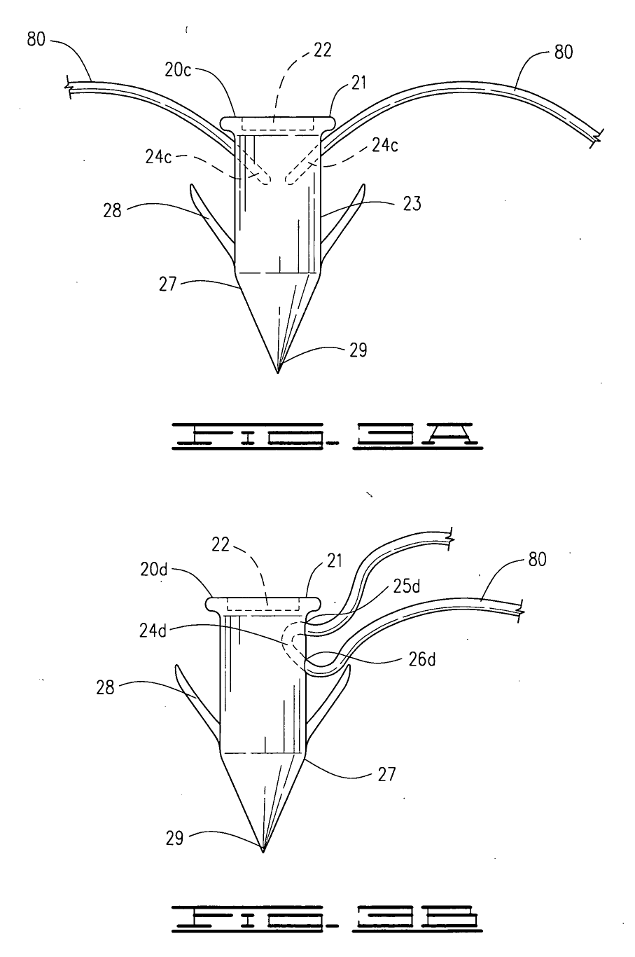

The single swedged pin 20a, shown in FIG. 1 and FIG. 2 of the drawings, includes a first end 82 of the suture 80 swedged into a single suture attachment orifice 24a. This single swedged pin 20a has the first end 82 of the suture 80 permanently attached or swedged into the single suture attachment 24a and may be used with another single swedged pin 24a attached to a second end 84 of the same suture 80 to form a fixed length suture staple device 10, or used with any other embodiment of a pin or pins 20, depending on the surgical connection being made.

The passage suture pin 20b, shown in FIG. 2 of the drawings, allows the suture 80 to be inserted or threaded through a downwardly displaced passage attachment orifice 24b. In this passage suture pin 20b, the suture 80 is not affixed to the passage suture pin 20b, but is adjustably threaded through the passage attachment orifice 24b, requiring the suture 80 to be fastened to another pin 20 or connected to the passage suture pin 20b by tying the suture 80 into a knot after adjustment to the length or tension of the suture 80.

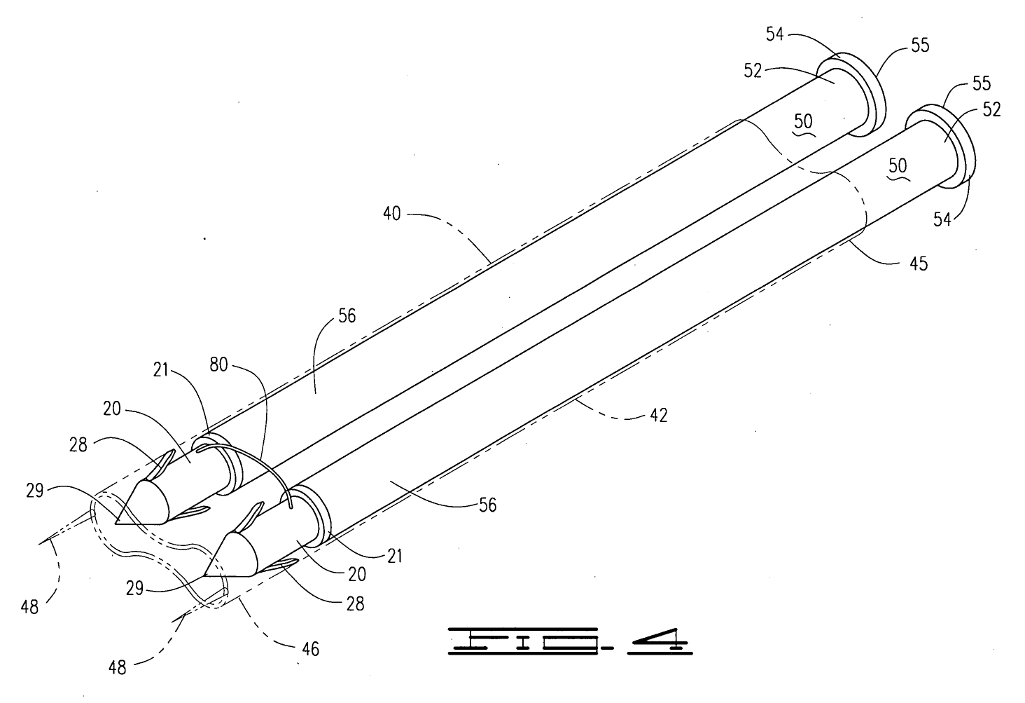

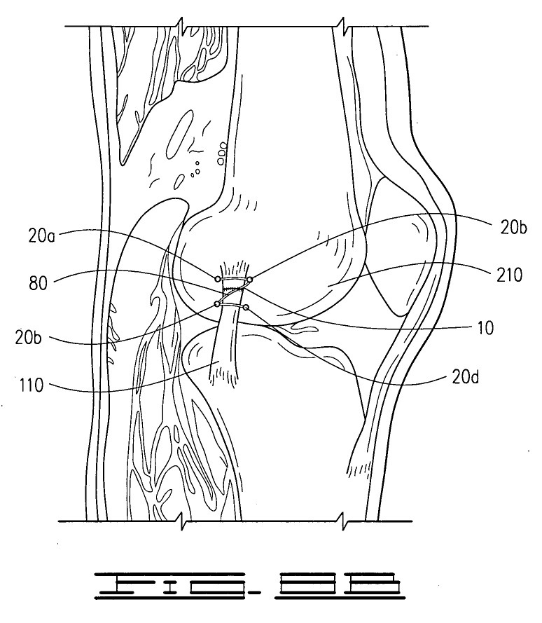

The double swedged pin 20c, shown in FIG. 3A of the drawings, has at least two sutures 80 swedged respectively into at least two single suture attachment orifices 24c. The double swedged pin 20c provides two independent sutures 80 to be connected to two or more other pins 20 or other connecting devices, and may also be utilized to secure soft tissue 100 in two or more manners and in multiple directions for reduction of forces on the soft tissue 100 by dispersion of the tension forces over having the soft tissue 100 attached in a single direction, FIG. 8B.

The adjustable locking passage pin 20d, shown in FIG. 3B of the drawings, includes an upwardly displaced locking passage attachment orifice 24d. The locking passage attachment orifice 24d allows for the suture 80 to be adjusted to length during the implant process into the bone when upward tension is applied to the suture emanating from an upper portion 25d of the locking passage attachment orifice 24d, but prohibits the length of the suture 80 from being adjusted when upward tension is applied to the suture 80 emanating from the lower portion 26d of the locking passage attachment orifice 24d. This adjustable locking passage pin 20d may be used in combination with any of the other embodiments of the pin 20a-20c, allowing the passing suture 80 to be locked into place after adjustment, in part, to length and tension with one pin 20, while still allowing for further adjustable connection to another pin 20.

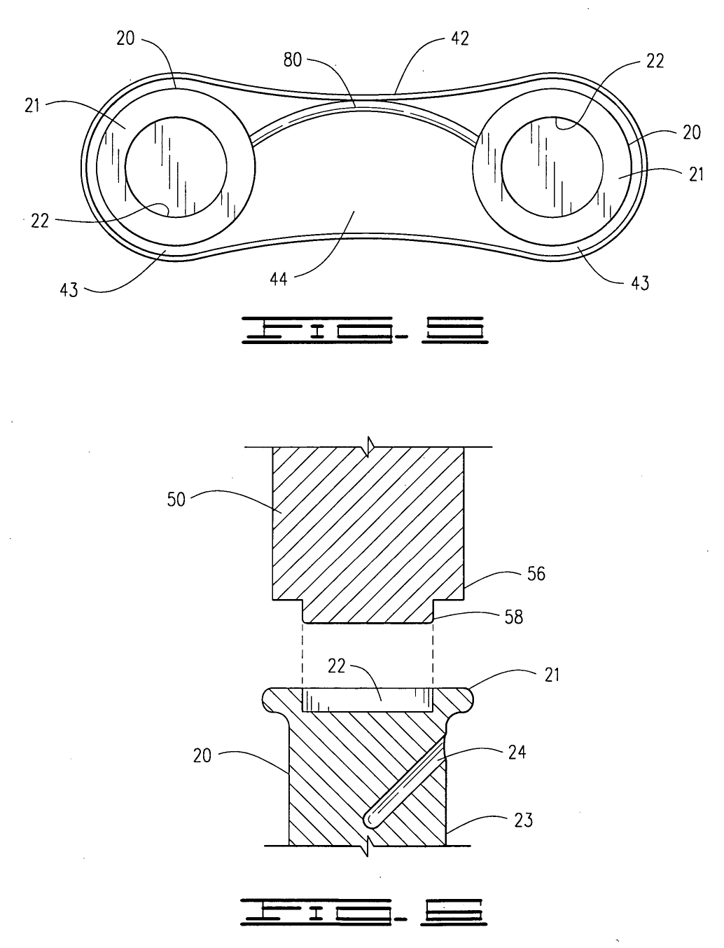

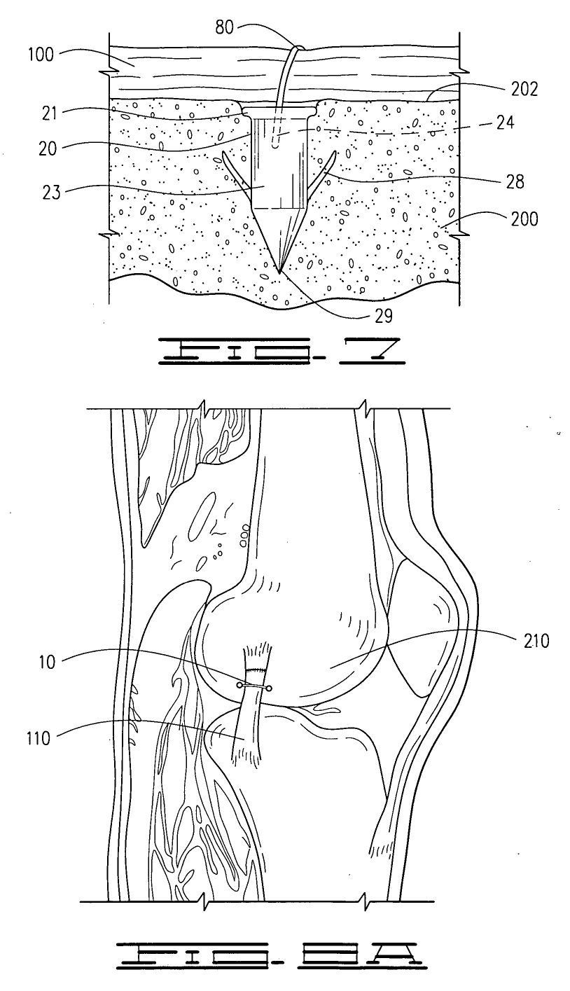

Insertion of the pins 20 into the bone 200 occurs during the surgical repair, most preferably during an arthroscopic surgical procedure, although application can also be applied during an open surgical procedure. In some cases, the soft tissue 100 is sutured together at a site of a tear prior to securing the soft tissue 100 to the bone 200, after which time the application of the suture staple device 10 occurs. In other cases, as surgeon may prefer to attach the soft tissue 100 to the bone 200 prior to repair of the torn soft tissue. Installation of the suture staple device 10 comprises the steps of loading at least one pin 20 attached to a suture 80 into the upper end 45 of at least one cylindrical insertion barrel 43 with the tapered point 29 directed towards the lower end 46 of the outer insertion tube 42, although the outer insertion tube 42 provides for the simultaneous installation of two pins 20, preferably connected by a suture 80 prior to installation, as indicated in FIGS. 4 and 5. The pins 20 are loaded into the upper end 45 providing a slight compression upon the lateral projecting bone anchor fins 28. Once the pins 20 are loaded into the cylindrical insertion barrels 43, the driving rods 50 are inserted into the open upper end 45 with the securing projection 58 of each driving rod 50 engaging each securing aperture 22 of the upper pin heads 21, as shown in FIG. 6. The lower end 46 of the outer insertion tube 42 is placed at the site of installation on the bone, with the at least two projecting anchor barbs 48 against the bone. The upper end 52 of the driving rods 50 are then struck, either individually or simultaneously with a surgical hammer, driving the pins 20 into the bone 200.

When installing the single swedged pin 20a or the double swedged pin 20c, the pin may be driven completely within the bone 200, with the upper pin head 21 slightly below the surface 202 of the bone 200, as shown in FIG. 7. However, when installing the passage suture pin 20b or the adjustable locking passage suture pin 20d, the tapered point 29 is inserted into the bone 200, with the respective passage attachment orifice 24b or locking passage attachment orifice 24d above the bone surface 202 until the suture 80 is properly tensioned within the respective orifice, after which the pins 24b, 24d are driven completely into the bone 200, with the upper pin heads 21 slightly below the surface 202 of the bone 200, shown in FIG. 7. Once driven into the bone, the lateral projecting bone anchor fins 28 inhibit the withdrawal of the pins 20 from the bone by being deployed outward and away from the shaft 23 of the pin 20 into the bone 200 in the event tension is applied to the pins 20 which would tend to extract the pins 20 from the bone 200.

Use of the attachment appliance 40 over other prior art attachment devices, especially those using some type of spring or air powered staple application device, is not recommended for use with the current suture staple device 10, because the surgeon must have control over the depth of the pin 20 into the bone 200 during the insertion and suture application process to insure that the soft tissue 200 is not strangled or cut during application of the suture staple device 10 to the bone 200. In addition, as the pins 20 may be partially inserted during theading or adjustment of the suture 80, only the lower end 27 of the pin 20 may be inserted within the bone 200 until the suture 80 is properly adjusted, with the upper pin head 21 of the pin 20 driven into the bone 200 after suture adjustment.

FIG. 8A illustrates an application of the suture staple device 10 to attach a soft tissue 100, in the illustration a ligament 110, to the lower femur 210 in a knee joint, without penetration of the ligament 110. FIG. 8B illustrates an application of the suture staple device 10 to attach a soft tissue, again a ligament 110, to the lower femur 210 utilizing a combination of different embodiments of the pin 20a-20d, as illustrated in FIG. 8B, the single swedged pin 20a, two passage suture pins 20b and an adjustable locking passage suture pin 20d using a single suture 80 in a Z-shaped pattern to secure the ligament 110 to the femur 210 without penetration of the ligament 110. Although not shown in FIGS. 8A and 8B, the pins 20 of the suture staple device 10 may also be driven through a soft tissue 100 into a bone 200 with the suture 80 or sutures tied to other pins 20 which may or may not be driven through the soft tissue 100.

Choices of materials for the pins 20 are preferably a biocompatible product including nytenol, stainless steel, titanium, or a non-deformable polymer. The suture 80 is preferably may of a flexible thread made of polyester, polypropylene, nylon or other biocompatible and nonabsorbable material, but could also be made from a thin cable of a polymeric material.

While the invention has been particularly shown and described with reference to a preferred embodiment thereof, it will be understood by those skilled in the art that changes in form and detail may be made therein without departing from the spirit and scope of the invention.

What is claimed is:

1. A suture staple device for the primary or secondary attachment of a soft tissue to a bone with or:
without requiring penetration of the soft tissue comprising:
at least two biocompatible pins including an expanded upper pin head defining a securing aperture, a shaft having at least one suture attachment orifice, and a lower end having two lateral projecting bone anchor fins, said lower end defining a tapered point;
at least one biocompatible flexible suture connected to said at least two pins; and
an application appliance further comprising,
an outer insertion tube having two parallel cylindrical insertion barrels connected by a linear suture passage, said outer insertion tube having an open upper end and an open lower end, said lower end having at least two projecting anchor barbs, and
two driving rods, each driving rod having an upper end defining an end cap with an upper striking surface and a lower end terminating into a securing projection adapted to securely fit within said securing aperture of said upper pin head, wherein said at least two pins are placed within said outer insertion tube with said lower end of each pin directed towards said lower end of said outer insertion tube and driven into said bone with said upper pin head completely embedded within said bone, said at least one suture retaining said soft tissue to said bone.

2. The suture staple device, as disclosed in claim 1, wherein at least one said pin is a single swedged pin with a single end of said suture swedged into a single suture attachment orifice which, when utilized with another single swedged pin attached to an opposing end of said same suture, provides said suture staple device with a fixed length suture.

3. The suture staple device, as disclosed in claim 1, wherein at least one pin is a passage suture pin to allow said suture to be inserted or threaded through a downwardly displaced passage attachment orifice, said suture not being affixed to said passage suture pin, but instead adjustably threaded through said passage attachment orifice, requiring said suture to be fastened to a second pin or connected to said passage suture pin by tying said suture to said passage suture pin.

4. The suture staple device, as disclosed in claim 1, wherein at least one pin is a double swedged pin having at least two sutures swedged respectively into at least two single suture attachment orifices, each of said at least two sutures emanating from said double swedged pin to be further connected to two or more other pins or other connecting devices, and also to be utilized to secure soft tissue in two or more manners and in multiple directions for reduction of forces on said soft tissue by dispersion of tension forces over having said soft tissue attached in a single direction.

5. The suture staple device as disclosed in claim 1, wherein said pin is an adjustable locking passage pin having an upwardly displaced locking passage attachment orifice allows for the suture to be adjusted to length during the implant process into the bone when upward tension is applied to the suture emanating from an upward portion of the locking passage orifice, but prohibiting said suture from being adjusted to length when upward tension is applied to the suture emanating from the lower portion of the locking passage orifice, said adjustable locking passage pin used in combination with any of the other pin, allowing said suture to be locked into place after adjustment, in part, to length and tension with one pin, while still allowing for further adjustable or fixed connection to another pin.

6. The suture staple device as disclosed in claim 1, wherein said pin is made from a material selected from a biocompatible material group consisting of nytenol, stainless steel, titanium or a non-deformable polymer and said suture is made from a material selected from a biocompatible flexible fabric group consisting of polyester, polypropylene, nylon or thin cable polymer.

7. A suture staple device for the primary or secondary attachment of a soft tissue to a bone with or without requiring penetration of the soft tissue comprising:
at least two biocompatible pins including an expanded upper pin head defining a securing aperture, a shaft having at least one suture attachment orifice, and a lower end having two lateral projecting bone anchor fins, said lower end defining a tapered point, wherein one of said at least two pins is a single swedged pin having a single suture attachment orifice;
at least one biocompatible flexible suture having two ends respectively connected to said at least two pins, with said one end of said suture being swedged into said single suture attachment orifice; and
an application appliance further comprising,
an outer insertion tube having two parallel cylindrical insertion barrels connected by a linear suture passage, said outer insertion tube having an open upper end and an open lower end, said lower end having at least two projecting anchor barbs, and
two driving rods, each driving rod having an upper end defining an end cap with an upper striing surface and a lower end terminating into a securing projection adapted to securely fit within said securing aperture of said upper pin head, wherein said at least two pins are placed within said outer insertion tube with said lower end of each pin directed towards said lower end of said outer insertion tube and driven into said bone with said upper pinhead completely embedded within said bone, said at least one suture retaining said soft tissue to said bone.

8. The suture staple device, as disclosed in claim 7, wherein said other pin of said at least two pins is a passage suture pin to allow said suture to be inserted or threaded through a downwardly displaced passage attachment orifice, said suture not being affixed to said passage suture pin, but instead adjustably threaded through said passage attachment orifice, requiring said suture to be fastened to said other of said at least two pins or connected to said passage suture pin by tying said suture to said passage suture pin.

9. The suture staple device, as disclosed in claim 7, wherein said other pin of said at least two pins is a double swedged pin having at least two sutures swedged respectively into at least two single suture attachment orifices, each of said at least two sutures emanating from said double swedged pin to be further connected to two or more other pins or other connecting devices, and also to be utilized to secure soft tissue in two or more manners and in multiple directions for reduction of forces on said soft tissue by dispersion of tension forces over having said soft tissue attached in a single direction.

10. The suture staple device as disclosed in claim 7, wherein said other pin of said at least two pins is an adjustable locking passage pin having an upwardly displaced locking passage attachment orifice allows for the suture to be adjusted to length during the implant process into the bone when upward tension is applied to the suture emanating from an upward portion of the locking passage orifice, but prohibiting said suture from being adjusted to length when upward tension is applied to the suture emanating from the lower portion of the locking passage orifice, said adjustable locking passage pin used in combination with any of the other pin, allowing said suture to be locked into place after adjustment, in part, to length and tension with one pin, while still allowing for further adjustable or fixed connection to another pin.

11. The suture staple device as disclosed in claim 7, wherein said pin is made from a material selected from a biocompatible material group consisting ofnytenol, stainless steel, titanium or a non-deformable polymer and said suture is made from a material selected from a biocompatible flexible fabric group consisting of polyester, polypropylene, nylon or thin cable polymer.

12. A method in installing the suture staple device using the application appliance, as disclosed in claim 7, comprising the steps of:
loading said at least one pin attached to said suture into said upper end of at least one said cylindrical insertion barrel, directing said lower end of said at least one pin towards said lower end of said outer insertion tube and slightly compressing said lateral projecting bone anchor fins towards said at least one pin;
inserting said at least one driving rod into said open upper end of said cylindrical insertion barrel loaded with said at least one pin;
positioning said securing projection on said driving rod within said securing aperture of said upper pin head of said at least one pin;
placing said lower end of said outer insertion tube at a location where said at least one pin is to be inserted into said bone;
positioning said at least two projecting anchor barbs against said bone stabilizing said outer insertion tube;
striking said upper end of said at least one driving rod to drive said at least one into said bone with the entire said upper pin head within said bone.

13. A method in installing the suture staple device using the application appliance, as disclosed in claim 7, comprising the steps of:
loading said two pins attached to said respective ends of said suture into said upper end of at least two said cylindrical insertion barrels, directing said lower ends of said tow pins towards said lower end of said outer insertion tube and slightly compressing said lateral projecting bone anchor fins towards each said two pins;
inserting said at least two driving rods into said open upper end of said cylindrical insertion barrels loaded with said two pins;
positioning said securing projections on said driving rods within said securing apertures of said upper pin heads of said two pins;
placing said lower end of said outer insertion tube at a location where said pins are to be inserted into said bone;
positioning said at least two projecting anchor barbs against said bone stabilizing said outer insertion tube;
striking said upper ends of said driving rods simultaneously or individually to drive each said pin into said bone, ultimately placing each said upper pin head within said bone.
